(19) United States
(12) Patent Application Publication
(10) Pub. No.: US 2004/0213798 A1
(43) Pub. Date: Oct. 28, 2004

(54) Spray-dried alum compositions

(75) Inventors: Yuh-Fun Maa, Millbrae, CA (US); Lu Zhao, Union City, CA (US); Steven J. Prestrelski, Mountain View, CA (US)

Correspondence Address: FOLEY AND LARDNER, SUITE 500, 3000 K STREET NW, WASHINGTON, DC, 20007, US

(73) Assignee: PowderJect Vaccines, Inc.

(21) Appl. No.: 10/852,740

(22) Filed: May 25, 2004

Related U.S. Application Data
(63) Continuation of application No. 09/590,777, filed on Jun. 8, 2000, now abandoned.

(51) Int. Cl. A61K 39/00

(52) U.S. Cl. 424/184.1

(57) ABSTRACT

A gel-forming free-flowing powder suitable for use as a vaccine is prepared by spray-drying an aqueous suspension that contains an antigen adsorbed to an aluminum salt adjuvant, a saccharide, an amino acid or a salt thereof, and a colloidal substance. Processes for forming these powder compositions are also described, as well as methods of using the compositions in a vaccination procedure.

### FIELD OF THE INVENTION

The invention relates generally to vaccine compositions. More specifically, the invention relates to vaccine compositions suitable for transdermal particle delivery from a needleless syringe system.

### BACKGROUND TO THE INVENTION

The ability to deliver pharmaceuticals agents into and through skin surfaces (transdermal delivery) provides many advantages over oral or parenteral delivery techniques. In particular, transdermal delivery provides a safe, convenient and noninvasive alternative to traditional administration systems, conveniently avoiding the major problems associated with oral delivery (e.g. variable rates of absorption and metabolism, gastrointestinal irritation and/or bitter or unpleasant drug tastes) or parenteral delivery (e.g. needle pain, the risk of introducing infection to treated individuals, the risk of contamination or infection of health care workers caused by accidental needle-sticks and the disposal of used needles).

However, despite its clear advantages, transdermal delivery presents a number of its own inherent logistical problems. Passive delivery through intact skin necessarily entails the transport of molecules through a number of structurally different tissues, including the stratum corneum, the viable epidermis, the papillary dermis and the capillary walls in order for the drug to gain entry into the blood or lymph system. Transdermal delivery systems must therefore be able to overcome the various resistances presented by each type of tissue.

In light of the above, a number of alternatives to passive transdermal delivery have been developed. These alternatives include the use of skin penetration enhancing agents, or “permeation enhancers,” to increase skin permeability, as well as non-chemical modes such as the use of iontophoresis, electroporation or ultrasound. However, these alternative techniques often give rise to their own unique side effects such as skin irritation or sensitization. Thus, the spectrum of agents that can be safely and effectively administered using traditional transdermal delivery methods has remained limited.

More recently, a novel transdermal drug delivery system that entails the use of a needleless syringe to fire powders (i.e., solid drug-containing particles) in controlled doses into and through intact skin has been described. In particular, commonly owned U.S. Pat. No. 5,630,796 to Bellhouse et al. describes a needleless syringe that delivers pharmaceutical particles entrained in a supersonic gas flow. The needleless syringe is used for transdermal delivery of powdered drug compounds and compositions, for delivery of genetic material into living cells (e.g., gene therapy) and for the delivery of biopharmaceuticals to skin, muscle, blood or lymph. The needleless syringe can also be used in conjunction with surgery to deliver drugs and biologics to organ surfaces, solid tumors and/or to surgical cavities (e.g., tumor beds or cavities after tumor resection). In theory, practically any pharmaceutical agent that can be prepared in a substantially solid, particulate form can be safely and easily delivered using such devices.

Vaccine compositions comprising an antigen adsorbed onto an aluminum salt adjuvant are well known. Spray-dried vaccine preparations comprising an immunogen adsorbed into an aluminum salt are disclosed in U.S. Pat. No. 5,902,565. Immediate-release preparations are described. Controlled- or delayed-release preparations are also described in which discrete immunogen-containing regions are present within a continuous matrix of biodegradable polymer.

The immediate-release preparations of U.S. Pat. No. 5,902,565 are prepared by spray-drying an aqueous suspension of aluminum salt-adsorbed immunogen. In the only Example, Example 1, in which such information is given, the resultant microspheres had a size range around 3 μm in diameter. The patent acknowledges that freeze-drying or lyophilisation of similar preparations has previously been described in U.S. Pat. No. 4,578,270 but that such freeze-drying or lyophilisation had a number of shortcomings. The most important shortcoming was the need to add large amounts of both dextran and protein so that partial retention of the aluminum gel structure could be achieved. This large addition of protein could however act to displace vaccine antigens from the aluminum gel and in addition would, in most cases, be immunogenic and as a result tend to swamp the immune response to the vaccine antigen.

According to U.S. Pat. No. 5,902,565 the gel-forming nature of aluminum gels is completely retained during spray-drying even in the absence of any other materials which could exert a stabilising effect (apart from minimal quantities of vaccine antigen, typically 1 to 10 μg/ml). Addition of water to the spray-dried powder was said to result in the instant formation of a typical gel, with sedimentation properties similar to the starting material.

EP-B-0130619 is also concerned with lyophilised vaccine preparations, in particular with a lyophilised preparation of a hepatitis B vaccine. The lyophilised preparation comprises an inactivated purified hepatitis B virus surface antigen absorbed to an aluminum gel and a stabiliser. The stabiliser is composed of at least one amino acid or salt thereof, at least one saccharide and at least one colloidal substance. Very low concentrations of aluminium salt adjuvant are used, typically less than 0.1% by weight.

### SUMMARY OF THE INVENTION

We investigated whether a gel-forming spray-dried powder of an aluminum salt could indeed be formed as described in U.S. Pat. No. 5,902,565. We were particularly interested in discovering whether such a spray-dried powder might be suitable for use in a needleless syringe. For that purpose, the spray-dried particles of aluminum salt should resuspend in water to form a gel. Minimal precipitates should form. Aggregation of the particles could adversely affect the immunogenicity of dry powder vaccines adjuvanted with an aluminum salt.

We found that spray drying a suspension of aluminum hydroxide or aluminum phosphate in water caused submicron particles of the aluminum salt to aggregate to larger particles in the resulting spray-dried powder. Upon reconstitution of this powder in water, these larger particles did not disintegrate into small particles. A gel suspension did not form. Rather, the aggregated particles of aluminum hydroxide or aluminum phosphate sedimented and precipitated out of the suspension.

Further experiments were carried out to determine if a spray-dried powder of an aluminum salt adjuvant could be formed that would be suitable for vaccine use when delivered by means of a needleless syringe. We found that a suitable powder could only be formed when an aluminum salt adjuvant was spray dried with a specific combination of other agents. Additionally, the aluminum salt adjuvant and other agents needed to be used in specific proportions.

Accordingly, the present invention provides a gel-forming free-flowing powder suitable for use as a vaccine, said powder being prepared by a process comprising spray-drying an aqueous suspension comprising:

(a) from 0.1 to 0.95% by weight of an aluminum salt adjuvant having an antigen adsorbed thereon;

(b) from 0.5 to 6% by weight of saccharide;

(c) from 0.1 to 2% by weight of an amino acid or salt thereof; and

(d) from 0.02 to 1% by weight of a colloidal substance.

Free-flowing powder compositions suitable for vaccine use can thus be produced. The compositions have well-defined particle size, density and mechanical properties which collectively are suitable for powders for transdermal delivery from a needleless syringe. The invention further provides:

a process for the preparation of a gel-forming free-flowing powder suitable for use as a vaccine, which process comprises spray-drying an aqueous suspension comprising:

(a) from 0.1 to 0.95% by weight of an aluminum salt adjuvant having an antigen adsorbed therein;

(b) from 0.5 to 6% by weight of a saccharide;

(c) from 0.1 to 2% by weight of an amino acid or salt thereof; and

(d) from 0.02 to 1% by weight of a colloidal substance.

a dosage receptacle for a needleless syringe, said receptacle containing an effective amount of a powder of the invention;

a needleless syringe which is loaded with a powder of the invention;

a vaccine composition comprising a pharmaceutically acceptable carrier or diluent and a powder of the invention;

a method of vaccinating a subject, which method comprises administering to the said subject an effective amount of a powder of the invention;

a gel-forming free-flowing powder suitable for use as a vaccine, which powder comprises:

(i) from 5 to 60% by weight of an aluminum salt adjuvant having an antigen adsorbed thereon;

(ii) from 25 to 90% by weight of a saccharide;

(iii) from 4.5 to 40% by weight of an amino acid or salt thereof; and

(iv) from 0.5 to 10% by weight of a colloidal substance.

### DETAILED DESCRIPTION OF THE PREFERRED EMBODIMENTS

Before describing the present invention in detail, it is to be understood that this invention is not limited to particularly exemplified compositions or process parameters as such may, of course, vary. It is also to be understood that the terminology used herein is for the purpose of describing particular embodiments of the invention only, and is not intended to be limiting.

All publications, patents and patent applications cited herein, whether supra or infra, are hereby incorporated by reference in their entirety.

It must be noted that, as used in this specification and the appended claims, the singular forms “a,” “an” and “the” include plural referents unless the content clearly dictates otherwise. Thus, for example, reference to “a particle” includes a mixture of two or more such particles, reference to “an excipient” includes mixtures of two or more such excipients, and the like.

A. Definitions

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

In describing the present invention, the following terms will be employed, and are intended to be defined as indicated below. By “antigen” is meant a molecule which contains one or more epitopes that will stimulate a host's immune system to make a cellular antigen-specific immune response or a humoral antibody response. Thus, antigens include polypeptides including antigenic protein fragments, oligosaccharides, polysaccharides and the like. Furthermore, the antigen can be derived from any known virus, bacterium, parasite, plants, protozoans or fungus, and can be a whole organism. The term also includes tumor antigens. Similarly, an oligonucleotide or polynucleotide which expresses an antigen, such as in DNA immunization applications, is also included in the definition of an antigen. Synthetic antigens are also included, for example polyepitopes, flanking epitopes and other recombinant or synthetically derived antigens (Bergmann et al (1993) Eur. J. Immunol. 23:2777-2781; Bergmann et al. (1996) J. Immunol. 157:3242-3249; Suhrbier, A. (1997) Immunol. and Cell Biol. 75:402-408; Gardner et al. (1998) 12^{th} World AIDS Conference, Geneva, Switzerland, June 28-Jul. 3, 1998).

The term “powder” as used herein refers to a composition that is comprised, or substantially consists of substantially solid particles that can be delivered transdermally using a needleless syringe device. The particles that make up the powder can be characterized on the basis of a number of parameters including, but not limited to, average particle size, average particle density, particle morphology (e.g. particle aerodynamic shape and particle surface characteristics) and particle penetration energy (P.E.).

The average particle size of the powders according to the present invention can vary widely and is generally from 0.1 to 250 μm, for example from 10 to 100 μm and more typically from 20 to 70 μm. The average particle size of the powder can be measured as a mass mean aerodynamic diameter (MMAD) using conventional techniques such as microscopic techniques (where particles are sized directly and individually rather than grouped statistically), absorption of gases, permeability or time of flight. If desired, automatic particle-size counters can be used (e.g. Aerosizer Counter, Coulter Counter, HIAC Counter, or Gelman Automatic Particle Counter) to ascertain the average particle size.

Actual particle density or “absolute density” can be readily ascertained using known quantification techniques such as helium pycnometry and the like. Alternatively, envelope (“tap”) density measurements can be used to assess the density of a powder according to the invention. The envelope density of a powder of the invention is generally from 0.1 to 25 g/cm³, preferably from 0.8 to 1.5 g/cm³.

Envelope density information is particularly useful in characterizing the density of objects of irregular size and shape. Envelope density is the mass of an object divided by its volume, where the volume includes that of its pores and small cavities but excludes interstitial space. A number of methods of determining envelope density are known in the art, including wax immersion, mercury displacement, water absorption and apparent specific gravity techniques. A number of suitable devices are also available for determining envelope density, for example, the GeoPyc™ Model 1360, available from the Micromeritics Instrument Corp. The difference between the absolute density and envelope density of a sample pharmaceutical composition provides information about the sample's percentage total porosity and specific pore volume.

Particle morphology, particularly the aerodynamic shape of a particle, can be readily assessed using standard light microscopy. It is preferred that the particles which make up the instant powders have a substantially spherical or at least substantially elliptical aerodynamic shape. It is also preferred that the particles have an axis ratio of 3 or less to avoid the presence of rod- or needle-shaped particles. These same microscopic techniques can also be used to assess the particle surface characteristics, e.g. the amount and extent of surface voids or degree of porosity.

Particle penetration energies can be ascertained using a number of conventional techniques, for example a metallized film P.E. test. A metallized film material (e.g. a 125 μm polyester film having a 350 Å layer of aluminum deposited on a single side) is used as a substrate into which the powder is fired from a needleless syringe (e.g. the needleless syringe described in U.S. Pat. No. 5,630,796 to Bellhouse et al) at an initial velocity of about 100 to 3000 m/sec. The metallized film is placed, with the metal coated side facing upwards, on a suitable surface.

A needleless syringe loaded with a powder is placed with its spacer contacting the film, and then fired. Residual powder is removed from the metallized film surface using a suitable solvent. Penetration energy is then assessed using a BioRad Model GS-700 imaging densitometer to scan the metallized film, and a personal computer with a SCSI interface and loaded with MultiAnalyst software (BioRad) and Matlab software (Release 5.1, The MathWorks, Inc.) is used to assess the densitometer reading. A program is used to process the densitometer scans made using either the transmittance or reflectance method of the densitometer. The penetration energy of the spray-coated powders should be equivalent to, or better than that of reprocessed mannitol particles of the same size (mannitol particles that are freeze-dried, compressed, ground and sieved according to the methods of commonly owned International Publication No. WO 97/48485, incorporated herein by reference).

The term “subject” refers to any member of the subphylum cordata including, without limitation, humans and other primates including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The methods described herein are intended for use in any of the above vertebrate species, since the immune systems of all of these vertebrates operate similarly.

The term “transdermal delivery” includes both transdermal (“percutaneous”) and transmucosal routes of administration, i.e. delivery by passage through the skin or mucosal tissue. See, e.g., Transdermal Drug Delivery: Developmental Issues and Research Initiatives, Hadgraft and Guy (eds.), Marcel Dekker, Inc., (1989); Controlled Drug Delivery: Fundamentals and Applications, Robinson and Lee (eds.), Marcel Dekker Inc., (1987); and Transdermial Delivery of Drugs, Vols. 1-3, Kydonieus and Bemer (eds.), CRC Press, (1987).

B. General Methods

The invention is concerned with gel-forming free-flowing powders suitable for use as vaccines. The powders are suitable for transdermal administration from a needleless syringe delivery system. As such, the particles which make up the powdered composition must have sufficient physical strength to withstand sudden acceleration to several times the speed of sound and the impact with, and passage through, the skin and tissue. The particles are formed by spray-drying an aqueous suspension comprising or, in some embodiments, consisting essentially of:

(a) from 0.1 to 0.95% by weight of an aluminum salt adjuvant having an antigen adsorbed therein;

(b) from 0.5 to 6% by weight of a saccharide;

(c) from 0.1 to 2% by weight of an amino acid or salt thereof; and

(d) from 0.02 to 1% by weight of a colloidal substance.

The aqueous suspension contains, as component (a), less than 1% by weight of the aluminum salt adjuvant having antigen adsorbed thereon. Preferably, the suspension contains from 0.2 or 0.3 to 0.6 or 0.75% by weight, preferably from 0.2 to 0.4% by weight, of aluminum salt adjuvant onto which antigen is adsorbed. The aluminum salt adjuvant is generally aluminum hydroxide or aluminum phosphate.

Any suitable antigen as defined herein may be employed. In this regard, the antigen of interest will preferably be associated with a pathogen, such as a viral, bacterial or parasitic pathogen, or the antigen may be a tumor-specific antigen or an antigen useful in breaking self-tolerance in autoimmune disorders such as in diabetes, lupus, arthritis, MS and in allergy.

For example, the antigen may be a viral antigen and may therefore be derived from members of the families Picomaviridae (e.g. polioviruses, etc.); Caliciviridae; Togaviridae (e.g. rubella virus, dengue virus, etc.); Flaviviridae; Coronaviridae; Reoviridae; Birnaviridae; Rhabodoviridae (e.g. rabies virus, etc.); Filoviridae; Paramyxoviridae (e.g. mumps virus, measles virus, respiratory syncytial virus, etc.); Orthomyxoviridae (e.g. influenza virus types A, B and C, etc.); Bunyaviridae; Arenaviridae; Retroviradae (e.g. HTLV-I; HTLV-II; HIV-1 and HIV-2); and simian immunodeficiency virus (SIV) among others.

Alternatively, viral antigens may be derived from papillomavirus (e.g. HPV); a herpesvirus; a hepatitis virus, e.g. hepatitis A virus, (HAV), hepatitis B virus (HBV), hepatitis C (HCV), the delta hepatitis virus (HDV), hepatitis E virus (HEV) or hepatitis G virus (HGV); and the tick-borne encephalitis viruses. See, e.g. Virology, 3rd Edition (W. K. Joklik ed. 1988); Fundamental Virology, 2nd Edition (B. N. Fields and D. M. Knipe, eds. 1991) for a description of these viruses.

Bacterial antigens for use in the invention can be derived from organisms that cause diphtheria, cholera, tuberculosis, tetanus, pertussis, meningitis and other pathogenic states, including Meningococcus A, B and C, Hemophilus influenza type B (HIB) and Helicobacter pylori. A combination of bacterial antigens may be provided, for example diphtheria, pertussis and tetanus antigens. Suitable pertussis antigens are pertussis toxin and/or filamentous haemagglutinin and/or pertactin, alternatively termed P69. An anti-parasitic antigen may be derived from organisms causing malaria and Lyme disease.

Antigens for use in the present invention can be produced using a variety of methods known to those of skill in the art. In particular, the antigens can be isolated directly from native sources, using standard purification techniques. Alternatively, whole killed, attenuated or inactivated bacteria, viruses, parasites or other microbes may be employed. Yet further, antigens can be produced recombinantly using known techniques. See, e.g., Sambrook, Fritsch & Maniatis, Molecular Cloning: A Laboratory Manual, Vols. I and II (D. N. Glover et. 1985).

Antigens for use herein may also be synthesised, based on described amino acid sequences, via chemical polymer syntheses such as solid phase peptide synthesis. Such methods are known to those of skill in the art. See, e.g. J. M. Stewart and J. D. Young, Solid Phase Peptide Synthesis, 2nd Ed., Pierce Chemical Co., Rockford, Ill. (1984) and G. Barany and R. B. Merrifield, The Peptides: Analysis, Synthesis, Biology, editors E. Gross and J. Meienhofer, Vol. 2, Academic Press, New York, (1980), pp. 3-254, for solid phase peptide synthesis techniques; and M. Bodansky, Principles of Peptide Synthesis, Springer-Verlag Berlin (1984) and E. Gross and J. Meienhofer, Eds., The Peptides: Analysis, Synthesis, Biology, supra, Vol. 1, for classical solution synthesis.

One or more saccharide may be present in the aqueous suspension as component (b). The saccharide content is typically 1.5 to 5% by weight, preferably 2 to 4% by weight. The saccharide may be a monosaccharide such as glucose, xylose, galactose, fructose, D-mannose or sorbose; a disaccharide such as lactose, maltose, saccharose, trehalose or sucrose; or a sugar alcohol such as mannitol, sorbitol, xylitol, glycerol, erythritol or arabitol.

One or more amino acids or amino acid salts is present in the aqueous suspension as component (c). Any physiologically acceptable amino acid salt may be employed. The salt may be an alkali or alkaline earth metal salt such as sodium, potassium or magnesium salt. The amino acid may be an acidic, neutral or basic amino acid. Suitable amino acids are glycine, alanine, glutamine, arginine, lysine and histidine. Monosodium glutamate is a suitable amino acid salt. The aqueous suspension for spray drying generally contains from 0.5 to 1.5% by weight, more preferably from 0.75 to 1.25% by weight, of the amino acid and/or amino acid salt.

The colloidal substance (d) is a divided substance incapable of passing through a semi-permeable membrane, comprised of fine particles which, in suspension or solution, fail to settle out. Suitable colloidal substances are disclosed in EP-B-0130619. Component (d) may be selected from polysaccharides such as dextran or maltodextran; hydrogels such as gelatin or agarose; or proteins such as human serum albumin. The substance may have a molecular weight of 500 to 80,000 or higher, for example from 1000 or 2000 to 30,000 or from 5,000 to 25,000. Component (d) is generally present in the aqueous suspension in an amount of from 0.05 to 0.5% by weight, preferably from 0.07 to 0.3% by weight.

The aluminum adjuvant salt having antigen adsorbed thereon and the saccharide, amino acid or salt thereof and colloidal substance are suspended in water. The aqueous suspension is spray dried. The spray-drying conditions are selected to enable the desired particles to be produced. The air inlet temperature, air outlet temperature, feed rate of the aqueous suspension, air flow rate, etc. can thus be varied as desired. Any suitable spray-drier may be used. The nozzle size may vary as necessary.

The spray-dried powder is free-flowing. It is capable of forming a gel when resuspended in water. No precipitate should form upon resuspension. Typically, a gel-like suspension without any precipitate is obtained after the powder has been added to distilled water (1:500 by weight) and shaken for three minutes. No precipitate may form after standing overnight, for example for 12 hours.

The presence of a precipitate, and the degree of agglomeration of the reconstituted gel formulation, is typically assessed by the ability of the reconstituted formulation to diffract a beam of light. The degree of agglomeration can also be quantitatively assessed by standard light microscopy and/or sedimentation. Another suitable test for particle agglomeration can be to determine particle size before and after reconstitution using any of a number of standard particle size determination techniques, e.g. laser-based or light obscuration.

The particles of the invention have a size appropriate for high-velocity transdermal delivery to a subject, typically across the stratum corneum or a transmucosal membrane. The mass mean aerodynamic diameter (MMAD) of the particles is from about 0.1 to 250 μm. The MMAD may be from 5 to 100 μm or from 10 to 1001 m, preferably from 10 to 701 m or from 20 to 70 μm. Generally, less than 10% by weight of the particles have a diameter which is at least 5 μm more than the MMAD or at least 5 μm less than the MMAD. Preferably, no more than 5% by weight of the particles have a diameter which is greater than the MMAD by 5 μm or more. Also preferably, no more than 5% by weight of the particles have a diameter which is smaller than the MMAD by 5 μm or more.

The particles have an envelope density of from 0.1 to 25 g/cm³, preferably from 0.8 to 1.5 g/cm³. While the shape of the individual particles may vary when viewed under a microscope, the particles are preferably substantially spherical. The average ratio of the major axis:minor axis is typically from 3:1 to 1:1, for example from 2:1 to 1:1.

A gel-forming free-flowing powder can thus be provided which is suitable for use as a vaccine. The proportions of the various components of the powder can be adjusting by adjusting the composition of the suspension that is spray-dried. However, the powder typically comprises, or in some embodiments consists essentially of:

(i) from 5 to 60%, for example from 7 to 50% such as from 10 to 30%, by weight of an aluminum salt adjuvant having an antigen adsorbed thereon;

(ii) from 25 to 90%, for example from 30 to 80% such as from 40 to 70%, by weight of a saccharride;

(iii) from 4.5 to 40%, for example from 7 to 30% such as from 10 to 20%, by weight of an amino acid or salt thereof; and

(iv) from 0.5 to 10%, for example from 0.8 to 6% such as from 1 to 3%, by weight of a colloidal substance.

A powder of the present invention is suitable for transdermal delivery from a needleless syringe delivery device. As well as appropriate average size and envelope density, the individual particles of the powder have a substantially spherical aerodynamic shape with a substantially uniform, nonporous surface. The particles will also have a particle penetration energy suitable for transdermal delivery from a needleless syringe device.

A detailed description of needleless syringe devices useful in this invention is found in the prior art, as discussed herein. These devices are referred to as needleless syringe devices and representative of these devices are the dermal PowderJect® needleless syringe device and the oral PowderJect® needleless syringe device (PowderJect Technologies Limited, Oxford, UK). By using these devices, an effective amount of the powder of the invention is delivered to the subject. An effective amount is that amount needed to deliver sufficient of the desired antigen to achieve vaccination. This amount will vary with the nature of the antigen and can be readily determined through clinical testing based on known activities of the antigen being delivered. The “Physicians Desk Reference” and “Goodman and Gilman's The Pharmacological Basis of Therapeutics” are useful for the purpose of determined the amount needed.

Needleless syringe devices for delivering particles were first described in commonly owned U.S. Pat. No. 5,630,796 to Bellhouse et al, incorporated herein by reference. Although a number of specific device configurations are now available, such devices are typically provided as a pen-shaped instrument containing, in linear order moving from top to bottom, a gas cylinder, a particle cassette or package, and a supersonic nozzle with an associated silencer medium. An appropriate powder (in the present case, a spray-dried powder of the invention) is provided within a suitable container, e.g., a cassette formed by two rupturable polymer membranes that are heat-sealed to a washer-shaped spacer to form a self-contained sealed unit. Membrane materials can be selected to achieve a specific mode of opening and burst pressure that dictate the conditions at which the supersonic flow is initiated. In operation, the device is actuated to release the compressed gas from the cylinder into an expansion chamber within the device. The released gas contacts the particle cassette and, when sufficient pressure is built up, suddenly breaches the cassette membranes sweeping the particles into the supersonic nozzle for subsequent delivery. The nozzle is designed to achieve a specific gas velocity and flow pattern to deliver a quantity of particles to a target surface of predefined area. The silencer is used to attenuate the noise produced by the membrane rupture.

A second needleless syringe device for delivering particles is described in commonly owned International Publication No. WO 96/20022. This delivery system also uses the energy of a compressed gas source to accelerate and deliver powdered compositions; however, it is distinguished from the system of U.S. Pat. No. 5,630,796 in its use of a shock wave instead of gas flow to accelerate the particles. More particularly, an instantaneous pressure rise provided by a shock wave generated behind a flexible dome strikes the back of the dome, causing a sudden eversion of the flexible dome in the direction of a target surface. This sudden eversion catapults a powdered composition (which is located on the outside of the dome) at a sufficient velocity, thus momentum, to penetrate target tissue, e.g., oral mucosal tissue. The powdered composition is released at the point of full dome eversion. The dome also serves to completely contain the high-pressure gas flow which therefore does not come into contact with the tissue. Because the gas is not released during this delivery operation, the system is inherently quiet. This design can be used in other enclosed or otherwise sensitive applications for example, to deliver particles to minimally invasive surgical sites.

In yet a further aspect of the invention, single unit dosages or multidose containers, in which the powder of the invention may be packaged prior to use, can comprise a hermetically sealed container enclosing a suitable amount of the powder that makes up a suitable dose. The powder can be packaged as a sterile formulation, and the hermetically sealed container can thus be designed to preserve sterility of the formulation until use. If desired, the containers can be adapted for direct use in the above-referenced needleless syringe systems.

Powders of the present invention can thus be packaged in individual unit dosages for delivery via a needleless syringe. As used herein, a “unit dosage” intends a dosage receptacle containing a therapeutically effective amount of a powder of the invention. The dosage receptacle typically fits within a needleless syringe device to allow for transdermal delivery from the device. Such receptacles can be capsules, foil pouches, sachets, cassettes or the like.

The container in which the powder is packaged can further be labeled to identify the composition and provide relevant dosage information. In addition, the container can be labeled with a notice in the form prescribed by a governmental agency, for example the Food and Drug Administration, wherein the notice indicates approval by the agency under Federal law of the manufacture, use or sale of the powder contained therein for human administration.

The actual distance which the delivered particles will penetrate a target surface depends upon particle size (e.g., the nominal particle diameter assuming a roughly spherical particle geometry), particle density, the initial velocity at which the particle impacts the surface, and the density and kinematic viscosity of the targeted skin tissue. In this regard, optimal particle densities for use in needleless injection generally range between about 0.1 and 25 g/cm³, preferably between about 8.9 and 1.5 g/cm³, and injection velocities generally range between about 100 and 3,000 m/sec. With appropriate gas pressure, particles having an average diameter of 10-70 μm can be accelerated through the nozzle at velocities approaching the supersonic speeds of a driving gas flow.

If desired, the needleless syringe systems can be provided in a preloaded condition containing a suitable dosage of the powder of the invention. The loaded syringe can be packaged in a hermetically sealed container, which may further be labeled as described above.

A number of novel test methods have been developed, or established test methods modified, in order to characterize performance of a needleless syringe device. These tests range from characterization of the powdered composition, assessment of the gas flow and particle acceleration, impact on artificial or biological targets, and measures of complete system performance. One, several or all of the following tests can thus be employed to assess the physical and functional suitability of the powder of the invention for use in a needleless syringe system.

Assessment of Effect on Artificial Film Targets

A functional test that measures many aspects of powder injection systems simultaneously has been designated as the “metallized film” or “penetration energy” (PE) test. It is based upon the quantitative assessment of the damage that particles can do to a precision thin metal layer supported by a plastic film substrate. Damage correlates to the kinetic energy and certain other characteristics of the particles. The higher the response from the test (i.e., the higher the film damage/disruption) the more energy the device has imparted to the particles. Either electrical resistance change measurement or imaging densitometry, in reflectance or transmission mode, provide a reliable method to assess device or formulation performance in a controllable and reproducible test.

The film test-bed has been shown to be sensitive to particle delivery variations of all major device parameters including pressure, dose, particle size distribution and material, etc. and to be insensitive to the gas. Aluminum of about 350 Angstrom thickness on a 125 μm polyester support is currently used to test devices operated at up to 60 bar.

Assessment of Impact Effect on Engineering Foam Targets

Another means of assessing particle performance when delivered via a needleless syringe device is to gauge the effect of impact on a rigid polymethylimide foam (Rohacell 5 IIG, density 52 kg/m³, Rohm Tech Inc., Malden, Mass.). The experimental set-up for this test is similar to that used in the metallized film test. The depth of penetration is measured using precision calipers. For each experiment a processed mannitol standard is run as comparison and all other parameters such as device pressure, particle size range, etc., are held constant. Data also show this method to be sensitive to differences in particle size and pressure. Processed mannitol standard as an excipient for drugs has been proven to deliver systemic concentrations in preclinical experiments, so the relative performance measure in the foam penetration test has a practical in vivo foundation. Promising powders can be expected to show equivalent or better penetration to mannitol for anticipation of adequate performance in preclinical or clinical studies. This simple, rapid test has value as a relative method of evaluation of powders and is not intended to be considered in isolation.

Particle Attrition Test

A further indicator of particle performance is to test the ability of various candidate compositions to withstand the forces associated with high-velocity particle injection techniques, that is, the forces from contacting particles at rest with a sudden, high velocity gas flow, the forces resulting from particle-to-particle impact as the powder travels through the needleless syringe, and the forces resulting from particle-to-device collisions also as the powder travels through the device. Accordingly, a simple particle attrition test has been devised which measures the change in particle size distribution between the initial composition, and the composition after having been delivered from a needleless syringe device.

The test is conducted by loading a particle composition into a needleless syringe as described above, and then discharging the device into a flask containing a carrier fluid in which the particular composition is not soluble (e.g., mineral oil, silicone oil, etc.). The carrier fluid is then collected, and particle size distribution in both the initial composition and the discharged composition is calculated using a suitable particle sizing apparatus, e.g., an AccuSizer®) model 780 Optical Particle Sizer. Compositions that demonstrate less than about 50%, more preferably less than about 20% reduction in mass mean diameter (as determined by the AccuSizer apparatus) after device actuation are deemed suitable for use in the needleless syringe systems described herein.

Delivery to Human Skin in vitro and Transepidermal Water Loss

For a powder performance test that more closely parallels eventual practical use, candidate powder compositions can be injected into dermatomed, full thickness human abdomen skin samples. Replicate skin samples after injection can be placed on modified Franz diffusion cells containing 32° C. water, physiologic saline or buffer. Additives such as surfactants may be used to prevent binding to diffusion cell components. Two kinds of measurements can be made to assess performance of the formulation in the skin.

To measure physical effects, i.e. the effect of particle injection on the barrier function of skin, the transepidermal water loss (TEWL) can be measured. Measurement is performed at equilibrium (about 1 hour) using a Tewameter TM 210® (Courage & Khazaka, Koln, Ger) placed on the top of the diffusion cell cap that acts like a ˜12 mm chimney. Larger particles and higher injection pressures generate proportionally higher TEWL values in vitro and this has been shown to correlate with results in vivo. Upon particle injection in vitro TEWL values increased from about 7 to about 27 (g/m² h) depending on particle size and helium gas pressure. Helium injection without powder has no effect. In vivo, the skin barrier properties return rapidly to normal as indicated by the TEWL returning to pretreatment values in about 1 hour for most powder sizes. For the largest particles, 53-75 μm, skin samples show 50% recovery in an hour and full recovery by 24 hours.

Delivery to Human Skin in vitro and Drug Diffusion Rate

To measure the formulation performance in vitro, the antigen component(s) of candidate powders can be collected by complete or aliquot replacement of the Franz cell receiver solution at predetermined time intervals for chemical assay using HPLC or other suitable analytical technique. Concentration data can be used to generate a delivery profile and calculate a steady state permeation rate. This technique can be used to screen formulations for early indication of antigen binding to skin, antigen dissolution, efficiency of particle penetration of stratum corneum, etc., prior to in vivo studies.

These and other qualatative and quantitative tests can be used to assess the physical and functional suitability of the present powders for use in a high-velocity particle injection device. It is preferred, though not required, that the particles of a powder have the following characteristics: a substantially spherical shape (e.g. an aspect ratio as close as possible to 1); a smooth surface; a suitable active loading content; less than 20% reduction in particle size using the particle attrition test; an envelope density as close as possible to the true density of the constituents (e.g. greater than about 0.8 g/ml); and a MMAD of about 20 to 70 μm with a narrow particle size distribution. The compositions are typically free-flowing (e.g. free-flowing after 8 hours storage at 50% relative humidity and after 24 hours storage at 40% relative humidity). All of these criteria can be assessed using the above-described methods, and are further detailed in the following publications, incorporated herein by reference. Etzler et al (1995) Part. Part. Syst. Charact. 12:217; Ghadiri, et al (1992) IFPRI Final Report, FRR 16-03 University of Surrey, UK; Bellhouse et al (1997) “Needleless delivery of drugs in dry powder form, using shock waves and supersonic gas flow,” Plenary Lecture 6, 21^{st}International Symposium on Shock Waves, Australia; and Kwon et al (1998) Pharm. Sci. suppl. 1 (1), 103.

A powder of the invention may alternatively be used to vaccinate a subject via other routes. For this purpose, the powder may be combined with a suitable carrier or diluent such as Water for Injections or physiologically saline. The resulting vaccine composition is typically administered by injection, for example subcutaneously or intramuscularly.

Whichever route of administration is selected, an effective amount of antigen is delivered to the subject being vaccinated. Generally from 50 ng to 1 mg and more preferably from 1 μg to about 50 μg of antigen will be useful in generating an immune response. The exact amount necessary will vary depending on the age and general condition of the subject to be treated, the particular antigen or antigens selected, the site of administration and other factors. An appropriate effective amount can be readily determined by one of skill in the art.

Dosage treatment may be a single dose schedule or a multiple dose schedule. A multiple dose schedule is one in which a primary course of vaccination may be with 1-10 separate doses, followed by other doses given at subsequent time intervals, chosen to maintain and/or reinforce the immune response, for example at 14 months for second dose and, if needed, a subsequent dose(s) after several months. The dosage regimen will also, at least in part, be determined by the need of the subject and be dependent on the judgement of the practitioner. Vaccination will of course generally be effected prior to primary infection with the pathogen against which protection is desired.

C. Experimental

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### REFERENCE EXAMPLE 1

A spray-dried immediate-release vaccine preparation was obtained according to the procedure described in U.S. Pat. No. 5,902,565. A formulation containing 5% by weight mannitol and 5% by weight aluminum phosphate (Adju-Phos) was spray dried using a bench-top spray dryer (Buchi 190). The spray-drying conditions were: inlet temperature=130° C.; outlet temperature=70° C., liquid feed rate=3 ml/min; atomizing airflow rate=500 1/hr; and a full scale of drying air. The free-flowing powder that was obtained had a particle size of about 10 μm. The powder was reconstituted in distilled water (1:500 by weight). The solution failed to form a gel with the suspended particles setting in 15 minutes. By optical microscopy, the particles after reconstitution maintained their shape and size, suggesting that the alum remained coagulated and didn't disintegrate.

### EXAMPLE 1

The following formulations were prepared by mixing the components listed in the Table below in 15 ml of distilled water:

| Formulation | Aluminum Salt | Mannitol | Glycine | Dextran |
|---|---|---|---|---|
| 1 (comparison) | 14.5 g of Alhydrogel¹⁾ | 322 mg | 131 mg | 17.5 mg |
| 2 (invention) | 2.5 g of Alhydrogel¹⁾ | 693 mg | 130 mg | 18 mg |
| 3 (comparison) | 15 g of Adju-Phos²⁾ | 438 mg | 173 mg | 16.9 mg |
| 4 (comparison) | 7.7 g of Adju-Phos²⁾ | 882 mg | 172 mg | 16.2 mg |

¹⁾Alhydrogel: 3% by weight aluminum hydroxide
²⁾Adju-Phos: 2% by weight aluminum phosphate

These formulations were spray dried using a Buchi 190 Mini-Spin Drier operating under the following conditions: air inlet temperature=130° C.; air outlet temperature=70° C.; Q liquid feed: setting 5; and Q atomising air: 500 1/hr. Drying air was set at the full scale. Free-flowing powders were obtained. Yields were as follows:

| Formulation | Powder yield (g) | % Yield | MMAD |
|---|---|---|---|
| 1 | 0.52 | 68.4 | 8-10 μm |
| 2 | 0.48 | 53.9 | 8-10 μm |
| 3 | 0.91 | 74.1 | 8-10 μm |
| 4 | 0.38 | 31 | 8-10 μm |

The composition of the powders obtained in relation to the solids content of the suspension subjected to spray drying was as follows:

| | | | | |
|---|---|---|---|---|
| | | | | Total |
| Al(OH)₃ | Mannitol | Glycine | Dextran | Solid |

| Formulation 1 | | | | | |
|---|---|---|---|---|---|
| Solid content in | 2.9 | 2.1 | 0.9 | 0.1 | 6 |
| suspension for | | | | | |
| spray drying (%) | | | | | |
| Powder content | 48.3% | 35.0% | 15.0% | 1.7% | |
| Formulation 2 | | | | | |
| Solid content in | 0.5 | 4.6 | 0.9 | 0.1 | 6.1 |
| suspension for | | | | | |
| spray drying (%) | | | | | |
| Powder content | 8.2% | 75.4% | 14.8% | 1.6% | |
| | | | | | Total |
| | AlPO₄ | Mannitol | Glycine | Dextran | Solid |
| Formulation 3 | | | | | |
| Solid content in | 4 | 2.9 | 1.2 | 0.1 | 8.2 |
| suspension for | | | | | |
| spray drying (%) | | | | | |
| Powder content | 48.8% | 35.4% | 14.6% | 1.2% | |
| Formulation 4 | | | | | |
| Solid content in | 1 | 5.9 | 1.1 | 0.1 | 8.1 |
| suspension for | | | | | |
| spray drying (%) | | | | | |
| Powder content | 12.3% | 72.8% | 13.6% | 1.2% | |

The spray dried powders were resuspended in distilled water. Specifically, each powder was added to distilled water (1:500 by weight) and shaken for 3 minutes. The resulting suspensions were examined for aggregation. Only Formulation 2 according to the invention formed a gel-like suspension without precipitate. The results are shown below:

Formulation 1: 32.59 mg of spray-dried powder was added to 1 ml of distilled water. A white precipitate formed after the resulting suspension has been allowed to stand overnight.

Formulation 2: 37.1 mg of spray-dried powder was added to 1 ml of distilled water. An off-white, grey, gel-like suspension formed. No precipitate was observed after the suspension had been allowed to stand overnight.

Formulation 3: 44.34 mg of spray-dried powder was added to 1 ml of distilled water. A white precipitate formed after the resulting suspension had been allowed to stand overnight.

Formulation 4: 29.4 mg of spray-dried powder was added to 1 ml of distilled water. A white precipitate formed after the resulting suspension had been allowed to stand overnight.

Accordingly, novel spray-dried powder compositions and methods for producing these compositions have been described. Although preferred embodiments of the subject invention have been described in some detail, it is understood that obvious variations can be made without departing from the spirit and the scope of the invention as defined by the appended claims.

What is claimed is:

1. A gel-forming free-flowing powder suitable for use as a vaccine, said powder being prepared by a process comprising spray-drying an aqueous suspension comprising:
(a) from 0.1 to 0.95% by weight of an aluminum salt adjuvant having an antigen adsorbed therein;
(b) from 0.5 to 6% by weight of a saccharide;
(c) from 0.1 to 2% by weight of an amino acid or salt thereof; and
(d) from 0.02 to 1% by weight of a colloidal substance.

2. A powder according to claim 1, wherein the aluminum salt adjuvant is aluminum hydroxide or aluminum phosphate.

3. A powder according to claim 1, wherein the antigen is a bacterial or viral antigen.

4. A powder according to claim 1, wherein the saccharide is a monosaccharide, disaccharide or sugar alcohol.

5. A powder according to claim 1, wherein the saccharide is selected from the group consisting of glucose, xylose, galactose, fructose, D-mannose, sorbose, lactose, maltose, saccharose, trehalose, sucrose, mannitol, sorbitol, xylitol, glycerin, glycerol, erythritol and arabitol.

6. A powder according to claim 1, wherein the amino acid is an acidic, neutral or basic amino acid.

7. A powder according to claim 1, wherein the amino acid or salt thereof is selected from the group consisting of glycine, alanine, glutamine, arginine, lysine, histidine and monosodium glutamate.

8. A powder according to claim 1, wherein the colloidal substance is selected from the group consisting of polysaccharides, hydrogels and proteins.

9. A powder according to claim 8, wherein the said substance is selected from the group consisting of dextran, maltodextran, gelatin, agarose and human serum albumin.

10. A powder according to claim 1, wherein the aqueous suspension comprises from 0.2 to 0.4% by weight of the aluminum salt adjuvant having antigen adsorbed thereon, from 2 to 4% by weight of the saccharide, from 0.75 to 1.25% by weight of the amino acid or salt thereof and from 0.07 to 0.3% by weight of the colloidal substance.

11. A powder according to claim 1, which comprises:
(i) from 7 to 50% by weight of the aluminum salt adjuvant having an antigen adsorbed therein,
(ii) from 30 to 80% by weight of the saccharide,
(iii) from 7 to 30% by weight of the amino acid or salt thereof, and
(iv) from 0.8 to 6% by weight of the colloidal substance.

12. A powder according to claim 1, having a mass mean aerodynamic diameter of from 10 to. 100 μm and an envelope density of from 0.8 to 1.5 g/cm³.

13. A powder according to claim 1, which forms a gel-like suspension without any precipitate after having been added to distilled water (1:500 by weight) and shaken for 3 minutes.

14. A process for the preparation of a gel-forming free-flowing powder suitable for use as a vaccines which process comprises spray-drying an aqueous suspension comprising:
(a) from 0.1 to 0.95% by weight of an aluminum salt adjuvant having an antigen adsorbed therein;
(b) from 0.5 to 6% by weight of a saccharide;
(c) from 0.1 to 2% by weight of an amino acid or salt thereof; and
(d) from 0.02 to 1% by weight of a colloidal substance.

15. A process according to claim 14, wherein the resultant spray-dried powder forms a gel-like suspension without any precipitate after having been added to distilled water (1:500 by weight) and shaken for 3 minutes.

16. A dosage receptacle for a needleless syringe, said receptacle containing an effective amount of a gel-forming free-flowing powder prepared by a process comprising spray-drying an aqueous suspension comprising:
(a) from 0.1 to 0.95% by weight of an aluminum salt adjuvant having an antigen adsorbed therein;
(b) from 0.5 to 6% by weight of a saccharide;
(c) from 0.1 to 2% by weight of an amino acid or salt thereof; and
(d) from 0.02 to 1% by weight of a colloidal substance.

17. A receptacle according to claim 16, wherein the receptacle is selected from the group consisting of capsules, foil pouches, sachets and cassettes.

18. A receptor according to claim 16, wherein the spray-dried powder forms a gel-like suspension without any precipitate after having been added to distilled water (1:500 by weight) and shaken for 3 minutes.

19. A needleless syringe which is loaded with a gel-forming free-flowing powder prepared by a process comprising spray-drying an aqueous suspension comprising:
(a) from 0.1 to 0.95% by weight of an aluminum salt adjuvant having an antigen adsorbed therein;
(b) from 0.5 to 6% by weight of a saccharide;
(c) from 0.1 to 2% by weight of an amino acid or salt thereof; and
(d) from 0.02 to 1% by weight of a colloidal substance.

20. A syringe according to claim 19, wherein the spray-dried powder forms a gel-like suspension without any precipitate after having been added to distilled water (1:500 by weight) and shaken for 3 minutes.

21. A vaccine composition comprising a pharmaceutically acceptable carrier or diluent and a gel-forming free-flowing powder prepared by a process comprising spray-drying an aqueous suspension comprising:
(a) from 0.1 to 0.95% by weight of an aluminum salt adjuvant having an antigen adsorbed therein;
(b) from 0.5 to 6% by weight of a saccharide;
(c) from 0.1 to 2% by weight of an amino acid or salt thereof; and
(d) from 0.02 to 1% by weight of a colloidal substance.

22. A vaccine composition according to claim 21, wherein the spray-dried powder forms a gel-like suspension without any precipitate after having been added to distilled water (1:500 by weight) and shaken for 3 minutes.

23. A method of vaccinating a subject, which method comprises administering to the said subject an effective amount of a gel-forming free-flowing powder prepared by a process comprising spray-drying an aqueous suspension comprising:
(a) from 0.1 to 0.95% by weight of an aluminum salt adjuvant having an antigen adsorbed therein;
(b) from 0.5 to 6% by weight of a saccharide;
(c) from 0.1 to 2% by weight of an amino acid or salt thereof; and
(d) from 0.02 to 1% by weight of a colloidal substance.

24. A method according to claim 23, wherein the powder is administered by a needleless syringe.

25. A method according to claim 23, wherein the powder is formulated with a pharmaceutically acceptable carrier or diluent.

26. A method according to claim 25, wherein the formulation is administered subcutaneously or intramuscularly.

27. A method according to claim 23, wherein the spray-dried powder forms a gel-like suspension without any precipitate after having been added to distilled water (1:500 by weight) and shaken for 3 minutes.

28. A gel-forming free-flowing powder suitable for use as a vaccine, which powder comprises:
(i) from 5 to 60% by weight of an aluminum salt adjuvant having an antigen adsorbed thereon;
(ii) from 25 to 90% by weight of a saccharide;
(iii) from 4.5 to 40% by weight of an amino acid or salt thereof; and
(iv) from 0.5 to 10% by weight of a colloidal substance.

29. A powder according to claim 28, which forms a gel-like suspension without any precipitate after having been added to distilled water (1:500 by weight) and shaken for 3 minutes.
